(19) United States
(12) Patent Application Publication
(10) Pub. No.: US 2008/0166645 A1
(43) Pub. Date: Jul. 10, 2008

(54) PYRANTHRONE TYPE COMPOUND, ORGANIC PHOTORECEPTOR, IMAGE FORMING METHOD AND IMAGE FORMING APPARATUS

(75) Inventors: Toyoko SHIBATA, Kanagawa (JP); Tomoko SAKIMURA, Tokyo (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA BUSINESS TECHNOLOGIES, INC., Tokyo (JP)

(21) Appl. No.: 11/873,032

(22) Filed: Oct. 16, 2007

(30) Foreign Application Priority Data
Dec. 18, 2006 (JP) 2006339642

(51) Int. Cl. G03G 13/06; G03C 1/73; C07C 49/617

(52) U.S. Cl. 430/72; 430/71; 430/97; 568/326

(57) ABSTRACT

An organic photoreceptor is described. The photosensitive layer contains a pyranthrone compound represented by Formula 1 which has been subjected to purification processing so as to have a mass reduction ratio D_{400/450} between 400° C. and 450° C. of not more than 2.0% in a thermogravimetric analysis;

D_{400/450}={(G₄₀₀−G₄₅₀)/Gᵢ}×100

wherein Gᵢ is a mass at 25° C., and G₄₀₀ and G₄₅₀ are each mass at 400° C. and 450° C., respectively,

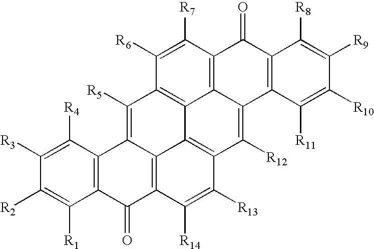

wherein R₁ through R₁₄ are each a hydrogen atom, an alkyl group, an alkoxy group, an aryl group, a halogen atom, a cyano group or a nitro group. The image forming method using the photoreceptor, and the pyranthrone compound are also disclosed.

This application is based on Japanese Patent Application No 2006-339642 filed on Dec. 18, 2006, in Japanese Patent Office, the entire content of which is hereby incorporated by reference.

### FIELD OF THE INVENTION

The invention provides a pyranthrone type compound having a new physical property which gives high sensitivity to a charge generation material of electrophotographic photoreceptor, and relates to an organic photoreceptor, and image forming method and an image forming apparatus each using the pyranthrene type compound, in detail to a pyranthrene type compound having a new physical property to be used for electrophotographic image formation in the field of copying machine and printer, and an organic photoreceptor, an image forming method and an image forming apparatus each using the pyranthrone type compound.

### TECHNICAL BACKGROUND

Recently, electrophotographic copying machines and printers are frequently applied in the field of printing and color printing. In the field of printing and color printing, high quality digital monochromatic or color image tends to be required. Corresponding to such the demand, it is proposed to form the high definition digital images by the use of laser light having short wavelength as the light source for exposing. It is the present circumstances, however, that the finally obtained electrophotographic image is not attained to sufficient high image quality even when the short wavelength laser light is used and the dot size of exposure is reduced so as to form a high definition electrostatic latent image on the electrophotographic photoreceptor.

The reason of such the affect is that the light sensitive property of the electrophotographic photoreceptor and the charging property of the toner of the developer do not satisfy the properties necessary for forming the fine dot latent image or toner image.

The electrophotographic photoreceptor using the organic photoreceptor, hereinafter also simply referred to as photoreceptor, developed for usual long wavelength laser light is inferior in the light sensitive property and the dot latent image cannot be clearly formed so that the reproducibility of the dot image tends to be degraded when the image exposure is carried out by the dot exposure of the short wavelength laser light with reduced diameter.

Hitherto, anthanthrone type pigments and pyranthrone type pigments are well known; cf. Patent Publication 1. There is no description in the publication regarding any specific treatment on the polycyclic quinone pigment such as the anthanthrone type pigments. Therefore, it is considered that the pigments available on the market are used in the publication. Sufficient sensitivity and high speed processing ability cannot be obtained by the light sensitive properties obtained by the use of the pigment available on the market when such the pigment is applied for the high speed printer and copier using the short wavelength laser which is expected to be developed near future.

It is also known that the polycyclic quinone type pigment is subjected to sublimation purification for raising the sensitivity thereof; cf. Patent Publication 2. However, the sublimation purification method described in the publication is a simplified one time sublimation purification treatment, and sufficient sensitivity and high speed processing ability cannot be obtained by the pigment prepared by this method in the high speed printer or the copier using the short wavelength laser.

Patent Publication 1: JP-A 2000-47408

Patent Publication 2: JP-A 57-67934

### DISCLOSURE OF THE INVENTION

The invention is attained for solving the above problems. An object of the invention is to provide a pyranthrone type compound having a new physical property to be used for an organic photoreceptor improved in the repeating usability and degradation of reproducibility of dot when a high density electrostatic latent image is formed on an organic photoreceptor by exposing light of 350 to 500 nm emitted from a semiconductor laser or a light emission diode, and to provide an organic photoreceptor, an image forming method and an image forming apparatus using the pyranthrone type compound.

As a result of investigation by the inventors on the above problems, it is found that the use of pyranthrone type compound having the new physical property which is improved in the light sensitive property for the short wavelength laser light which is used as the charge generation material of organic photoreceptor is effective for forming an electrophotographic image improved in the sensitivity, remaining potential and dot reproducibility for forming a high definition electrostatic latent image on the organic photoreceptor by imagewise exposure by a semiconductor laser or a light emission diode emitting light having a wavelength of from 350 to 500 nm, and attain the present invention.

The organic photoreceptor using the pyranthrone type compound, an image forming method using the photoreceptor and the pyranthrone type compound are described.

An organic photoreceptor has a photosensitive layer provided on an electroconductive substrate, and the photosensitive layer contains a pyranthrone compound represented by the following Formula 1 which has been subjected to a purification processing so as to have a mass reduction ratio D_{400/450} between 400° C. and 450° C. defined by the following expression of not more than 2.0% in the thermogravimetric analysis;

D_{400/450}={(G₄₀₀−G₄₅₀)/Gᵢ}×100

in the above, Gᵢ is the mass at the initial time of the measurement (room temperature, 25° C.), and G₄₀₀ and G₄₅₀ are each the mass at 400° C. and 450° C., respectively.

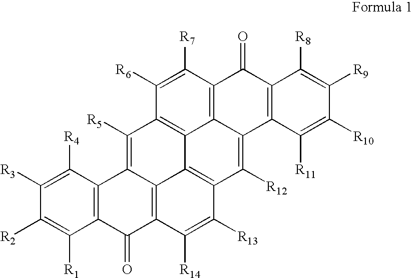

In Formula 1, R₁ through R₁₄ are each a hydrogen atom, an alkyl group, an alkoxy group, an aryl group, a halogen atom, a cyano group or a nitro group.

Preferable examples of the purification process is multi-step sublimation purification, train sublimation purification, a heat treatment in a high-boiling solvent and an acid paste treatment.

The mass reduction ratio D_{400/450} is preferably not more than 1.8%. The mass reduction ratio D_{400/450} is more preferably not more than 1.2%, and most preferably 1.0%.

In the Formula 1 R₁ through R₁₄ are preferably each a hydrogen atom, an alkyl group, an alkoxy group, an aryl group or a halogen atom group.

An image forming method comprises the steps of

exposing for forming an electrostatic latent image on an organic photoreceptor by using a semiconductor laser or a light emission diode emitting light having a wavelength of from 350 to 500 nm as a writing light source, and

developing for developing the electrostatic latent image to form a toner image,

and the organic photoreceptor described above is employed.

An image forming apparatus having an exposing means for forming an electrostatic latent image on an organic photoreceptor by using a writing light source of a semiconductor laser of a light emission diode emitting light having a wavelength of from 350 to 500 nm and a developing means for developing the electrostatic latent image to a toner image, wherein the organic photoreceptor described above is used.

A pyranthrone compound represented by the Formula 1 which has a mass reduction ratio D_{400/450} between 400° C. and 450° C. defined by the following expression of not more than 2.0% in the thermogravimetric analysis;

D_{400/450}={(G₄₀₀−G₄₅₀)/Gᵢ}×100

in the above, Gᵢ is the mass at the initial time of the measurement (room temperature, 25° C.), and G₄₀₀ and G₄₅₀ are each the mass at 400° C. and 450° C., respectively.

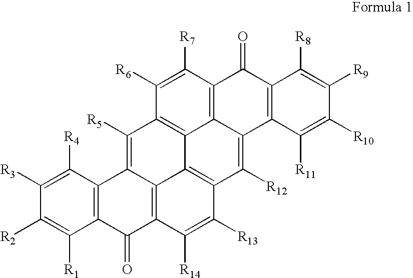

In Formula 1, R₁ through R₁₄ are each a hydrogen atom, an alkyl group, an alkoxy group, an aryl group, a halogen atom, a cyano group or a nitro group.

The pyranthrone type compound described above wherein the pyranthrone type compound is obtained by multi-step sublimation purification.

The pyranthrone type compound described above wherein the pyranthrone type compound is obtained by train sublimation purification.

The pyranthrone type compound described above wherein the pyranthrone type compound is obtained by heat treatment in a high-boiling solvent.

The pyranthrone type compound described above wherein the pyranthrone type compound is obtained by an acid paste treatment.

In the electrophotographic image forming system using short wavelength laser light, attenuation of electric potential per unit of light amount can be raised, repeating usability can be improved, sharp small dot can be formed so as to be able to form an electrophotographic image improved in the reproducibility of dot by applying the organic photoreceptor, image forming method and the image forming apparatus of the invention.

### BRIEF DESCRIPTION OF THE DRAWINGS

FIG. 1 is a schematic drawing of an image forming apparatus in which the function of the invention is included.

FIG. 2 is a cross section of a color image forming apparatus showing an embodiment of the invention.

FIG. 3 is a cross section of a color image forming apparatus using the organic photoreceptor of the invention.

### THE PREFERABLE EMBODIMENT OF THE INVENTION

This invention is described.

The pyranthrone type compound of the invention is a compound represented by a chemical structure represented by the following Formula 1 and has a mass reduction ratio D_{400/450} between 400° C. and 450° C. defined by the following expression of not more than 2.0%, preferably not more than 1.8% and more preferably not more than 1.2% in the thermogravimetric analysis.

D_{400/450}={(G₄₀₀−G₄₅₀)/Gᵢ}×100

In the above, Gᵢ is the mass at the initial time of the measurement (room temperature, 25° C.), and G₄₀₀ and G₄₅₀ are each the mass at 400° C. and 450° C., respectively.

The organic photoreceptor comprises a photosensitive layer provided on an electroconductive substrate and the photosensitive layer contains a pyranthrone type compound represented by the foregoing Formula 1 and has been subjected to purification processing so as to have a mass reduction ratio D_{400/450} between 400° C. and 450° C. defined by the following expression of not more than 2.0% in the thermogravimetric analysis.

D_{400/450}={(G₄₀₀−G₄₅₀)/Gᵢ}×100

In the above, Gᵢ is the mass at the initial time of the measurement (room temperature, 25° C.), and G₄₀₀ and G₄₅₀ are each the mass at 400° C. and 450° C., respectively.

In the invention, attenuation of electric potential per unit of light amount can be raised, repeating property can be improved, sharp small dot can be formed so as to be able to form an electrophotographic image improved in the reproducibility of dot in the electrophotographic image forming system using short wavelength laser light by using the pyranthrone type compound having the foregoing thermogravimetric property as the charge generation material of the organic photoreceptor.

The invention are described more in detail below.

The pyranthrone type compound of the invention has a mass reduction ratio D_{400/450} between 400° C. and 450° C. defined by the following expression of not more than 2.0% in the thermogravimetric analysis

D_{400/450}={(G₄₀₀−G₄₅₀)/Gᵢ}×100

In the above, Gᵢ is the mass at the initial time of the measurement (room temperature, 25° C.), and G₄₀₀ and G₄₅₀ are each the mass at 400° C. and 450° C., respectively.

The mass reduction ratio D_{400/450} is a reduction ratio of the mass of the pyranthrone type compound when the temperature of the compound is raised from 400° C. to 450° C. The ratio can be measured by the following condition.

Approximately 5 mg of the sample is heated from room temperature at a rate of 10° C. per minute under nitrogen stream of 100 ml/min and the mass when the temperature of the sample attained at 400° C. and that at 450° C. are measured and the reduction rate is determined from the difference of the measured values of the mass.

It is considered that the conventionally known pyranthrone type compound contains large amount of impurity volatilizable near the subliminal point thereof, and many impurity levels are caused in the pigment when large amount of the impurity is contained in the pigment of pyranthrone type compound so as to degrade the sensitivity and the repeating property of the photoreceptor.

The pyranthrone type compound usually used for the electrophotographic photoreceptor also contains considerable amount of the impurity. The impurity contains ingredients volatilizable at relative low temperature such as less than 400° C. The invention is based on the observation by the inventors that the amount of the ingredients volatilizable at a high temperature of from 400° C. to 450° C. near the sublimation temperature largely influences on the properties of the electrophotographic photoreceptor. The impurities volatilizable at a relatively low temperature of less than 450° C. can be removed by a suitable purifying method so that the amount of the impurity can be reduced. However, the influence of the impurity volatilizable near the sublimation point is larger in the electrophotographic photoreceptor than that of the entire amount of the impurity.

The pyranthrone type compound relating to the invention represented by Formula 1 and having a mass reduction ratio D_{400/450} of not more than 2.0% displays large attenuation value of potential per unit exposure amount and good repeating property and can form sharp dot latent images having small diameter so that an electrophotographic image improved in the dot reproducibility is obtained. The mass reduction ratio is more preferably not more than 1.0%.

Concrete examples of compound represented by Formula 1 are listed below.

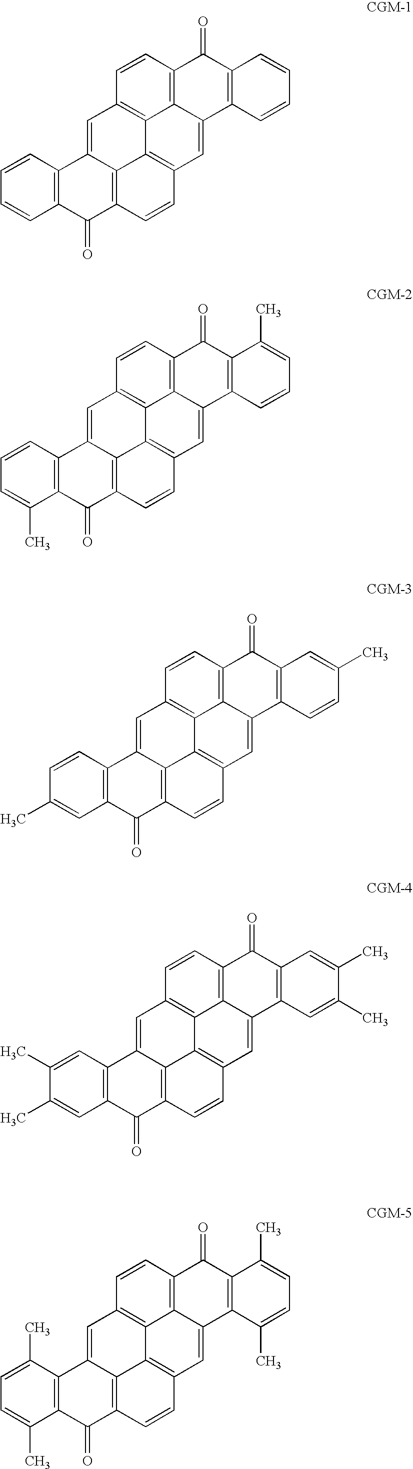

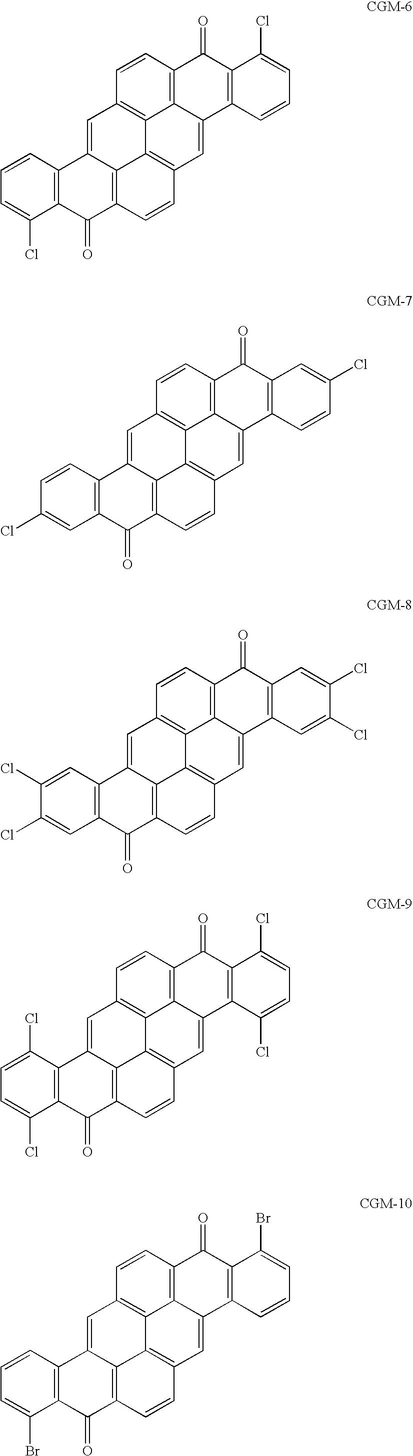

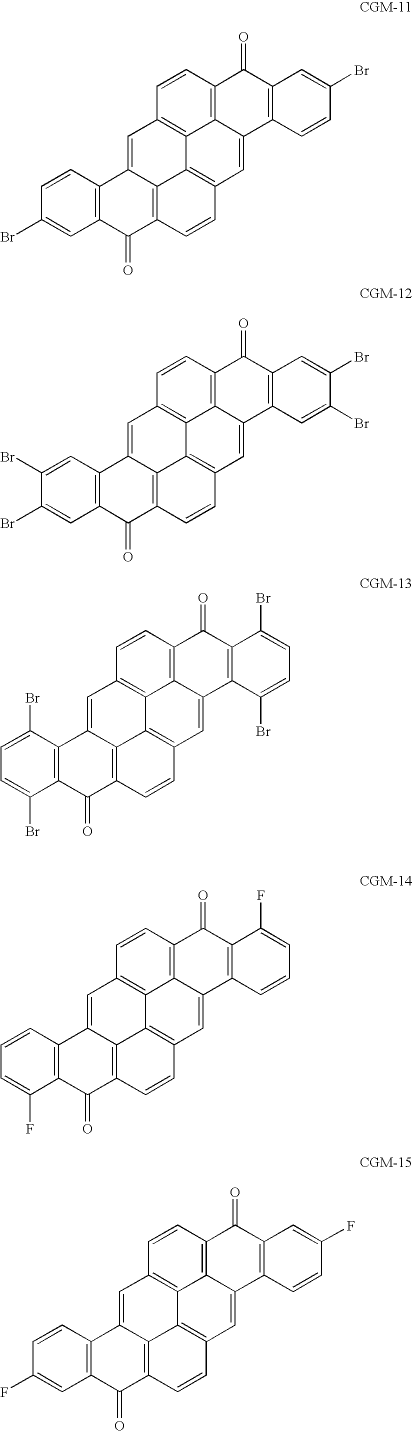

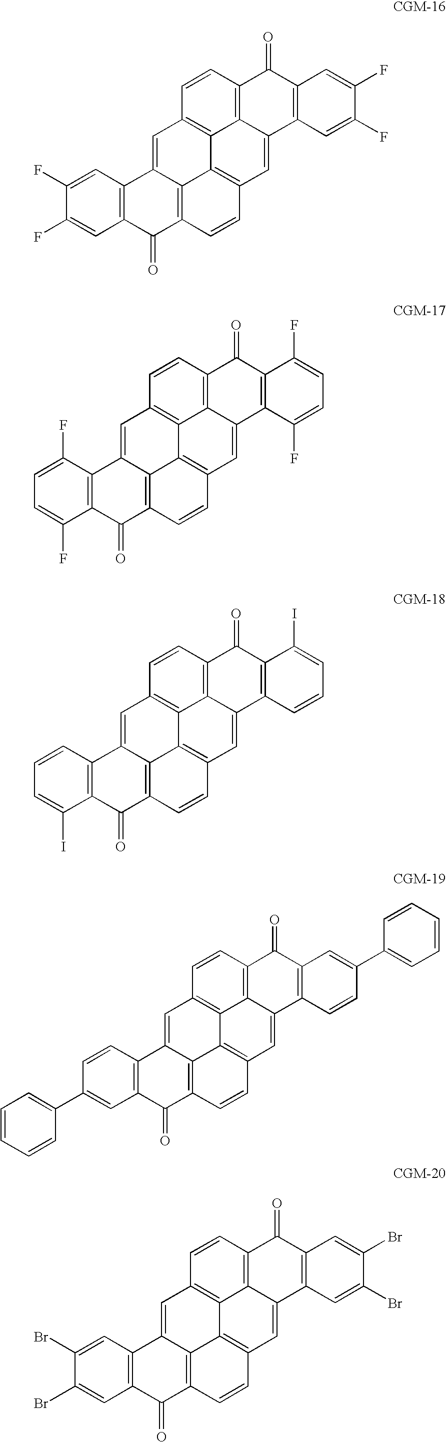

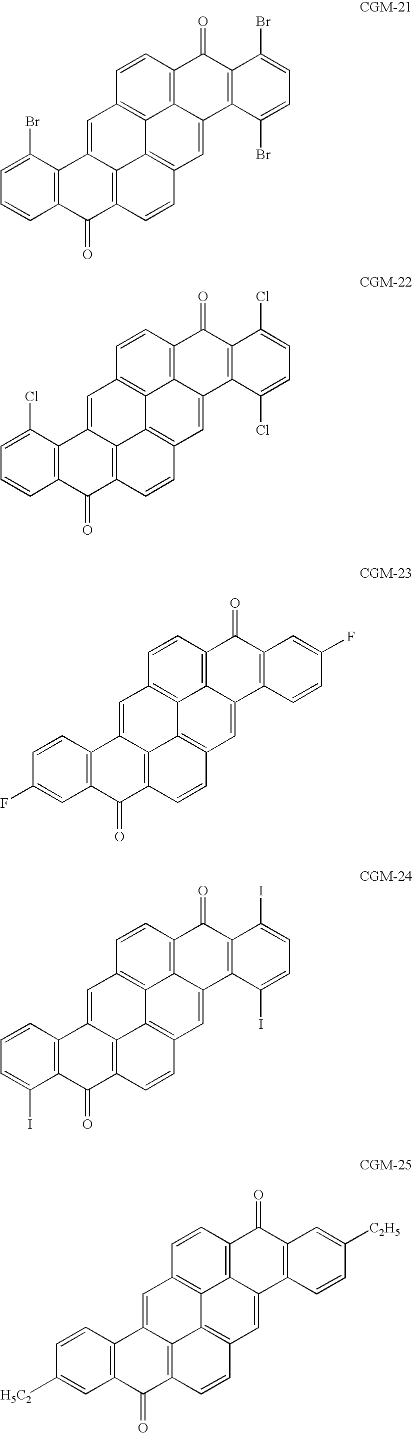

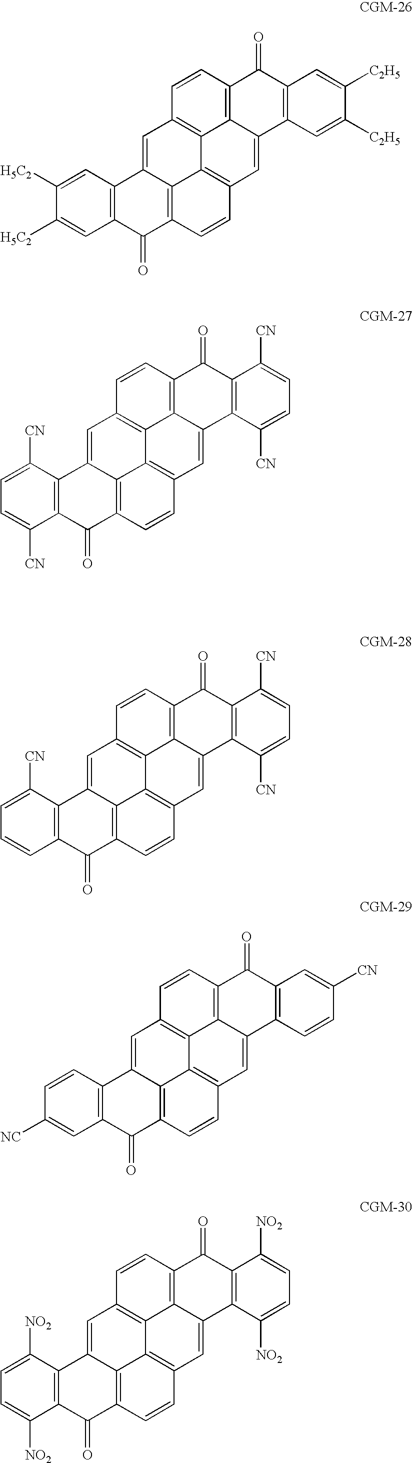

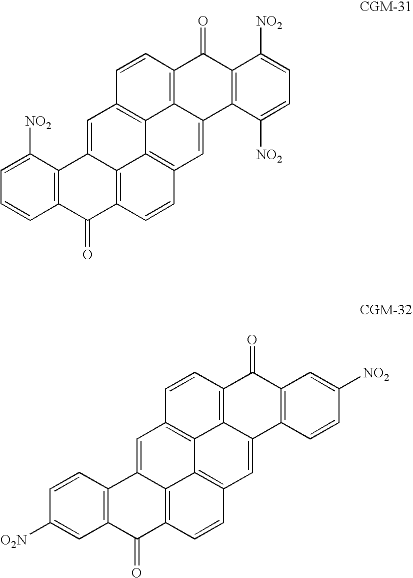

Synthesizing example of the compound represented by Formula 1 relating to the invention is described below.

Synthesizing example 1 (CGM-13)

Into a 500 ml of four-mouth flask, 15 g of pyranthrone, 150 g of chlorosulfuric acid and 0.75 g of iodine were charged and sufficiently dissolved. Into the resultant solution, 24 g of bromine was dropped. The system was gradually heated by 80 to 85° C. and made to react for 5 hours.

When the interior temperature of the flask was lowered by room temperature, the content was poured into a 3 L beaker containing 2 L of ice. After sufficient stirring, the resultant precipitate was filtered and washed until the filtrate of washing water becomes neutral. The washed material was dried to obtain 23 g of crude substance.

The purification method for obtaining the pyranthrone type compound having the mass reduction ratio of not more than 0.2% is described in the following a to d (purification treatment a to d for pyranthrone compound) using the pyranthrone compound (crude substance) obtained in the above Synthesizing Example 1.

(a): Pyranthrone Type Compound Obtained by a Multi-Step Purification

The multi-step sublimation purification is a purification process including two or more sublimation processes. On the first step, an effective amount such as 1 to 10% by weight of the substance is sublimated on a first substrate at a temperature slightly higher than the sublimation point of the substance. Then the sublimation temperature is raised within the range of from 10 to 100° C. on a second step so as to sublimate the substance on a second substrate. Thus highly purified pigment containing no volatile impurity and decomposed impurity can be obtained. The process may contain three or more steps according to circumstances.

### Purification Example 1

### Concrete Example of Multi-Step Sublimation Purification

Five grams of the pyranthrone type compound (CGM-13) obtained in Synthesizing Example 1 was put into a crucible and the interior pressure of chamber of the sublimation apparatus was reduced by 133.3 Pa to 13.3 Pa. Then the temperature of the crucible was raised by 420° C. and held for 10 minutes and then heating was stopped. The pressure in the chamber was made to atmosphere pressure when the temperature of the crucible lowered by 200° C. or less and 0.5 g of sublimated substance (first step sublimated SCM-13) was collected from the collection substrate. After that, the pressure in the sublimation apparatus was reduced by about 133.3 Pa to 13.3 Pa and the temperature of the crucible was heated at 450° C. for 2 hours. After cooling, 4.2 g of sublimated substance of pyranthrone type compound (second step sublimated CGM-13) deposited on the collector substrate was obtained. The mass reduction, ratio of thus purified CGM-13 was 0.5%.

(b): Pyranthrone Type Compound Obtained by Fractional Sublimation Purification

In the fractional sublimation purification, the pigment is heated at a temperature of T1 at the first position for volatizing the pigment and the volatile impurity, and the vapor of the pigment is condensed at the second position held at a temperature T2 lower than T1 and then the vapor of the volatile impurity is condensed at the third position held at a temperature T3 lowered than T2. Nonvolatile impurity remains at the first position where the starting substance is placed and the purified pigment separated from the volatile impurity can be obtained. The fractional sublimation method includes known purification method such as the train sublimation.

### Purification Example 2

### Concrete Example of the Fractional Sublimation Purification

Five grams of the pyranthrone type compound CGM-13 obtained in Synthesizing Example 1 was put into a PIREX® glass tube and the tube was set in a furnace in which temperature gradient of from about 480° C. to about 20° C. was formed along the length of the tube (the temperature gradient of about 48° C. to about 20° C. was formed in the length of 1 m). The pressure in the glass tube was reduced to approximately 133.3 Pa to 13.3 Pa and the position where pyranthrone type compound to be purified was placed was heated by about 480° C. Thus formed vapor was moved to the low temperature side and condensed, and 4.4 g of the sublimated substance of pyranthrone compound CGM-13 condensed at the portion between the about 300° C. and 420° C. was obtained. The mass reduction ratio of thus purified CGM-13 was 0.24%.

(c): Pyranthrone Compound Obtained by Heating Treatment in High-Boiling Solvent

In the heating treatment in high-boiling solvent, the non-purified pigment is heated in a high-boiling solvent having a boiling point of not less than 150° C. and recrystallized or washed for removing the impurity having high solubility in the high-boiling solvent. As the high-boiling solvent preferably used in the invention, o-dichlorobenzene, 1-chloronaphthalene, nitrobenzene, quinoline and sulfolane can be cited.

### Purification Example 3

### Concrete Example of Heating Treatment in High-Boiling Solvent

Five grams of pyranthrone type compound CGM-13 obtained in Synthesis Example 1 was put into a crucible and the pressure in the sublimation chamber was reduced by about 133.3 Pa to 13.3 Pa, then the temperature of the crucible is raised by 450° C. and held for 2 hours. After cooled, the pressure in the chamber was made to atmosphere pressure and 4.5 g of sublimated substance adhering on the collector substrate was obtained. One point zero gram of the sublimated substance was suspended in 100 ml of quinoline and heated at 200° C. for 1 hour and then filtered and washed by acetone and then by methanol, and dried to obtain purified CGM-13. The mass reduction ratio of thus obtained CGM-13 was 0.51%.

(d): Pyranthrone Type Compound Obtained by Acid Paste Treatment

Acid paste treatment is a treatment in which the pigment is dissolved in a strong acid such as sulfuric acid, chlorosulfuric acid and trifluoroacetic acid and poured into water for simultaneously forming fine particles, removing water-soluble inorganic impurity and accelerating amorphousness the pigment particle. Such the treatment is frequently applied for phthalocyanine pigment. Sulfuric acid or chlorosulfuric acid is preferably used for the acid paste treatment of the pyranthrone type compound of the invention.

Therefore, the crystals after washing and filtration usually contain water in an amount of from 5 to 10 times by weight of the pigment and are obtained in a form of wet cake or water-containing paste because the particles of the pigment treated by the acid paste treatment are very fine. The objective pigment can be obtained by drying the wet cake or the water-containing paste.

### Purification Example 4

### Concrete Example of Acid Paste Treatment

Five grams of pyranthrone type compound CGM-13 obtained in Synthesis Example 1 was put into a crucible and the pressure in the sublimation chamber was reduced by about 133.3 Pa to 13.3 Pa, then the temperature of the crucible is raised by 450° C. and held for 2 hours. After cooled, the pressure in the chamber was made to atmosphere pressure and 4.5 g of sublimated substance adhering on the collector substrate was obtained. Then 1.0 g of the sublimated substance was dissolved in 30 ml of trifluorosulfuric acid and the resultant solution was dropped into 500 ml of water. Precipitated pigment was filtered and washed by suspending in 200 ml of water. The washing was repeated until the electroconductivity of the water used for washing come down to 100 μS/cm or less. Thus obtained wet cake was dried to obtain purified CGM-13. The mass reduction ratio of thus obtained CGM-13 was 0.76%.

The organic photoreceptor of the invention is an organic photoreceptor containing the pyranthrone type compound represented by Formula 1 as the charge generation material. The constitution of the organic photoreceptor containing such the charge generation material is described below.

In this invention, the organic electrophotographic photoreceptor is defined as an organic electrophotographic photoreceptor containing an organic compound having at least one of the charge generation function and the charge transfer function. The charge generation function and the charge transfer function are essential functions for constituting the electrophotographic photoreceptor. The organic photoreceptor includes organic photoreceptors such as those constituted by organic charge generation materials or organic charge transfer materials and those containing a polymer complex having the charge generation function and the charge transfer function.

The constitution of the organic photoreceptor of the invention is not specifically limited as long as the photoreceptor contains the compound represented by Formula 1, and the following constitutions can be exemplified;

1) A charge generation layer and a charge transfer layer are successively provided as the photosensitive layer on an electroconductive substrate,

2) A charge generation layer, a first charge transfer layer and a second charge transfer layer are successively provided as the photosensitive layer on an electroconductive substrate,

3) A single photosensitive layer containing the charge transfer material and the charge generation material is formed on an electroconductive substrate,

4) A charge transfer layer and a charge generation layer are successively provided as the photosensitive layer on an electroconductive substrate, and

5) A surface protective layer is provided on the photosensitive layer of each of the photoreceptors 1) to 5).

The photoreceptor having any of the above constitutions are applicable. The surface layer of the photoreceptor is a layer contacting with air, and the photosensitive layer is the surface layer when a single layer type photosensitive layer is provided on the electroconductive substrate, and the surface protective layer is the outermost surface layer when the single layer type or the multi-layer type photosensitive layer is provided on the electroconductive substrate. In the invention, the above constitution 2) is most preferable. A under coat layer (intermediate layer) may be provided on the electroconductive substrate in previous to the formation of the photosensitive layer even when the photoreceptor has any constitution.

The charge transfer layer is a layer having a function of transfer the charge carrier generated in the charge generation layer by light exposure to the surface of the organic photosensitive layer, and the charge transfer function can be confirmed by detecting photoconductivity of the sample formed by piling the charge generation layer and the charge transfer layer on the electroconductive substrate.

The constitution of the organic photoreceptor is described below principally referring the constitution 1).

Electroconductive Substrate

Both of sheet-shaped and cylinder-shaped electroconductive substrates are applicable for the photoreceptor, and the cylindrical one is preferable for making compact the image forming apparatus.

The cylindrical electroconductive substrate is a cylindrical substrate necessary for endlessly forming images by rotation thereof, and one having a straightness of not more than 0.1 mm and a shaking of not more than 0.1 mm is preferable. Suitable image is difficultly formed when the linearity and the leaning are exceeds the above range.

A drum of metal such as aluminum and nickel, a plastic drum on which an electroconductive material such as aluminum, tin oxide and indium oxide is vapor deposited and a paper-plastic drum on which an electroconductive material is coated are usable as the electroconductive material. The relative resistivity of the electroconductive substrate is preferably not more than 10³Ω·cm at room temperature. The aluminum substrate is most preferable for the electroconductive substrate of the photoreceptor of the invention. One containing another ingredient such as manganese, zinc and magnesium additionally to aluminum may be used as the aluminum substrate.

Intermediate Layer

In the invention, an intermediate layer is preferably provided between the electroconductive substrate and the photosensitive layer.

It is preferable that the intermediate layer to be used in the invention contains an N-type semiconductor particle. The N-type semiconductor particle is a particle in which the principle charge carrier is an electron. The intermediate layer comprising an insulation binder containing the N-type semiconductor particles has characteristics that it effectively blocks positive hole injection from the substrate and shows less blocking of electron from the photosensitive layer, since the principal charge carrier in the N-type semiconductor particle is electron.

Titanium oxide (TiO₂) and zinc oxide (ZnO₂), particularly titanium oxide, are preferably used as the N-type semiconductor particle.

As the N-type semiconductor particle, fine particle having a number average primary particle diameter of from 3 to 200 nm is used and the average particle size of from 5 nm to 100 nm is particularly preferred. The number average primary particle diameter is an average value of diameter in Ferre's direction determined by observing 100 primary particles which are randomly selected from the image of the fine particles enlarged in 10,000 times by a transmission electron micrometer and analyzing by an image analysis. The N-type semiconductor particles having a number average primary particle diameter of less than 3 nm are difficultly dispersed in the binder of the intermediate layer and tend to form coagulated particles so that the remaining potential tends to be raised since the coagulated particles affect as charge traps. On the other hand, the N-type semiconductor particles having a number average primary particle diameter of more than 200 nm tends to cause large irregularity on the surface of the intermediate layer so that the dot image tends to be degraded by such the irregularity on the surface. Moreover, the N-type semiconductor particles having a number average primary particle diameter of more than 200 nm are easily precipitated in the dispersion and tend to form coagulated particles so that the dot images tend to be degraded.

The crystal shape of the titanium oxide particle includes anatase-type, rutile-type, brucite-type and amorphous-type. Among them, rutile-type titanium oxide pigment and anatase-type titanium oxide pigment are most preferable since such the pigments raise rectification ability of the charge passing through the intermediate layer, namely the mobility of electron can be raised, the charge potential can be stabilized and the remaining potential can be inhibited and the degradation of the dot image can be prevented.

The N-type semiconductor particle treated on the surface by a polymer containing methylhydrogen siloxane unit is preferable. The polymer containing methylhydrogen siloxane unit having a molecular weight of from 1,000 to 20,000 displays high surface treatment effect. Therefore, the rectification ability of the N-type semiconductor particle is raised, so that occurrence of black spots can be inhibited and sufficient reproducibility of dot image can be obtained by the use of the intermediate layer containing such the N-type semiconductor particles.

The polymer containing methylhydrogen siloxane unit is preferably a copolymer of a structural unit of —(HSi(CH₃)O)—and another structural unit namely another siloxane unit. As the other siloxane unit, a dimethylsiloxane unit, a methylethylsiloxane unit, a methylphenylsiloxane unit, and a diethylsiloxane unit are preferable and dimethylsiloxane is particularly preferable. The ratio of the methylhydrogen siloxane unit in the copolymer is from 10 to 99 mole-percent and preferably from 20 to 90 mole-percent.

The methylhydrogen siloxane copolymer may be any of a random copolymer, a block copolymer and a graft copolymer, and the random copolymer and the block copolymer are preferable. The copolymer ingredient other than the methylhydrogen siloxane may be one, two or more.

The intermediate layer coating liquid prepared for forming the intermediate layer comprises a binder resin and a dispersing solvent additionally to the N-type semiconductor particles such as the surface treated titanium oxide.

The ratio of the N-type semiconductor particles in the intermediate layer is preferably from 1.0 to 2.0 times of the volume of the binder resin in the intermediate layer. The rectification ability of the intermediate layer is raised by the use of the N-type semiconductor particle in the intermediate layer in such the high concentration so that the raising in the remaining potential and the degradation of the dot image can be effectively prevented even when the thickness of the intermediate layer is made thick and suitable organic photoreceptor can be prepared.

Polyamide resin is preferably used for sufficiently dispersing the particles as the binder resin for dispersing the particles to form the intermediate layer, and the following polyamide resins are particularly preferred.

An alcohol-soluble polyamide resin is preferable for the binder resin of the intermediate layer. As the binder resin of the intermediate layer of the organic photoreceptor, a resin having high solubility in solvent is required for forming the intermediate layer having uniform thickness. A copolymerized polyamide resin having a chemical structure which has few carbon chains between the amide bonds such as 9-Nylon is known as the alcohol-soluble polyamide resin. Moreover, the following polyamides can be preferably used other than the above resin.

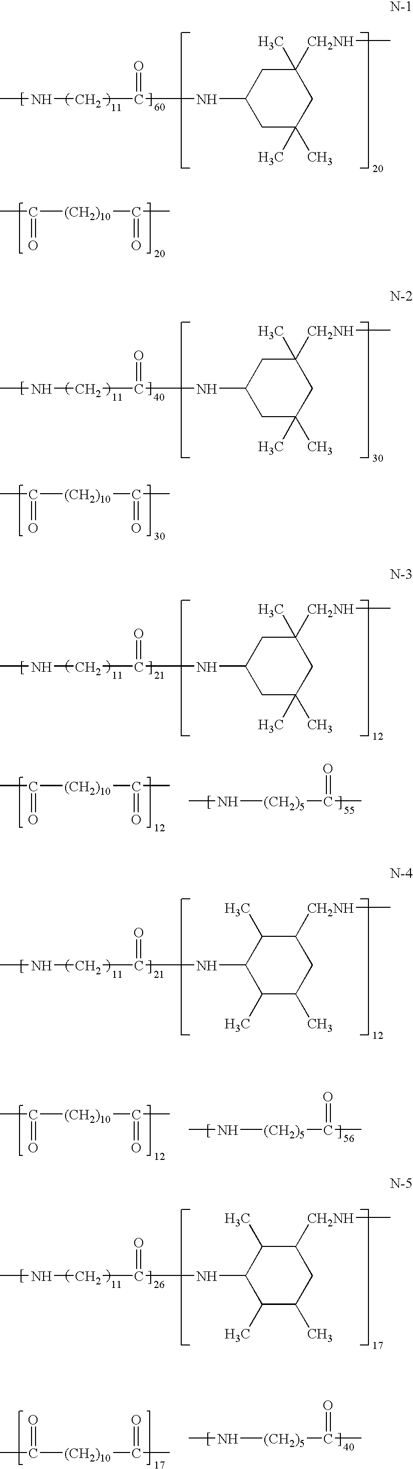

The number average molecular weight of the polyamide resin is preferably from 5,000 to 80,000, and more preferably from 10,000 to 60,000. When the number average molecular weight is less than 5,000, the thickness uniformity of the intermediate layer is degraded so that the effect of the invention is difficultly realized. On the other hand, when the molecular weight is more than 80,000, the solubility of the resin in the solvent tends to be lowered and resin coagula tend to be formed in the intermediate layer so that the black spots and degradation of the dot image tend to be caused.

A part of the polyamide resin is marketed, for example, under the trade name of VESTAMELT X1010 and X4685, manufactured by Daicel-Degussa Ltd., which can be produced by common synthesizing method of polyamide.

An alcohol having 2 to 4 carbon atoms such as ethanol, n-propyl alcohol, iso-propyl alcohol, n-butanol, t-butanol and sec-butanol is preferable as the solvent for dissolving the polyamide resin to prepare the coating liquid, which is superior in the dissolving ability to the polyamide and the coating suitability of the coating liquid. The ratio of such the solvent in the whole solvent is from 30 to 100%, preferably from 40 to 100%, and more preferably from 50 to 100%, by weight. Methanol, benzyl alcohol, toluene, methylene chloride, cyclohexanone and tetrahydrofuran are usable as a co-solvent giving good result by using together with the above solvent.

The thickness of the intermediate layer is preferably from 0.3 to 10 μm. A thickness of intermediate layer of less than 0.5 μm tends to cause black spots and degradation of dot image. When the thickness exceeds 10 μm, the remaining potential tends to be raised and dot image tends to be degraded. The thickness of intermediate layer is more preferably from 0.5 to 5 μm.

It is preferable that the intermediate layer is substantially electric non-conductive. The non-conductive layer has a layer having a volume resistance of not less than 1×10⁸Ω·cm. The volume resistance of the intermediate layer and the protective layer in the invention is preferably from 1×10⁸ to 1×10¹⁵Ω·cm, more preferably from 1×10⁹ to 1×10¹⁴Ω·cm, and further preferably from 2×10⁹ to 1×10¹³Ω·cm. The volume resistance can be measured by the following method.

Measuring condition: According to JIS C2318-1975

Measuring apparatus: Hiresta IP manufactured by Mitsubishi Chemical Corporation.

Measuring probe: HRS

Applying voltage: 500 V

Environmental condition: 30±2° C., 80±5 RH %

When the volume resistance is less than 1×10⁸Ω·cm, the blocking ability of the intermediate layer is lowered, occurrence of black spots is increased and the potential holding ability of the organic photoreceptor is degraded so that sufficient image quality cannot be obtained. When the volume resistivity exceeds 1×10¹⁵Ω·cm, the remaining potential caused by repeated image formation tends to be increase so that sufficient image quality cannot be obtained.

Photoreceptive Layer

The layer constitution of the photoreceptor of the invention is preferably a function separating constitution in which the function of the photosensitive layer is separated to a charge generation layer and a charge transfer layer though a single layer constitution may be applied, in which the charge generation function and the charge transfer function are possessed by one layer. By the function separating constitution, the remaining potential caused by repeatedly use can be controlled to low and another photographic property can be easily controlled so as to suit for purpose of use. In the negatively charging photoreceptor, a constitution is preferable, in which the charge generation layer is provided on the intermediate layer (CGL) and the charge transfer layer (CTL) is provided on the charge generation layer.

The constitution of the photoreceptor of the function separated negatively charging photoreceptor is described bellow.

Charge Generation Layer

In the organic photoreceptor, the charge generation material of the pyranthrone type compound is used, which has high sensitivity within the range of from 350 nm to 500 nm. Another charge generation material may be used according to necessity additionally to the above charge generation material. An azo pigment, a perylene pigment and a polycyclic quinine pigment are cited as the pigment to be used together with the pyranthrone type compound.

Reins can be used as the binder of the charge generation material (CGM) in the charge generation layer. A formal resin, a butyral resin, a silicone resin, a silicone-modified butyral resin and a phenoxy resin can be cited as the most preferable resin. The ratio of the charge generation material to the resin binder is preferably 20 to 600 parts by weight per 100 parts by weight of the binder resin. The increasing in the remaining potential caused by repeating use can be lowered by the use of such the resins. The thickness of the charge generation layer is preferably 0.3 μm to 2 μm.

Charge Transfer Layer

The charge transfer layer may be constituted by plural charge transfer layers and the outermost or surface charge transfer layer contains inorganic fine particles such as silica or alumina.

The charge transfer layer contains the charge transfer material (CTM) and a binder for dispersing or dissolving the CTM and forming the layer. Other than the above, an additive such as an antioxidant may be added according to necessity additionally to the inorganic fine particle.

A positive hole transfer type (P-type) charge transfer material can be used as the charge transfer material. For example, a triphenylamine derivative, a hydrazone compound, a styryl compound, a benzidine compound and a butadiene compound can be used. These charge transfer materials are usually dissolved in a suitable binder resin and made into the layer state.

A thermoplastic resin and a thermocurable resin are usable as the binder resin to be used in the charge transfer layer. Examples of the resin include a polystyrene, an acrylic resin, a methacrylic resin, a vinyl chloride resin, a vinyl acetate resin, a polyvinyl butyral resin, an epoxy resin, a polyurethane resin, a phenol resin, a polyester resin, an alkyd resin, a polycarbonate resin, a silicone resin, a melamine resin and a copolymer containing two or more recurring units of the above resins, and a organic polymer semiconductor such as poly-N-vinylcarbazole. Among them, polycarbonate resin is most preferable, which is small in the water absorption and suitable in the dispersing ability for CTM and the electrophotographic properties.

The ratio of the charge transfer material to the binder resin is from 50 to 200 parts by weight to 100 parts by weight of the binder.

The total thickness of the charge transfer layer is preferably from 10 to 30 μm. When the total thickness is less than 10 μm, sufficient latent image potential on the occasion of developing is difficultly obtained so that the lowering in the image density and the reproducibility of dot image tends to be caused. When the thickness exceeds 30 μm, diffusion of the charge carrier (diffusion of the charge carrier generated in the charge generation layer) is increased so that the reproducibility of dot image tends to be degraded. When the charge transfer layer is constituted by plural layers, the thickness of the surface charge transfer layer is preferably from 1.0 to 8.0 μm.

n-Butylamine, diethylamine, ethylenediamine, iso-propanolamine, triethanolamine, triethylenediamine, N,N-dimethylformamide, acetone, methyl ethyl ketone, methyl isopropyl ketone, cyclohexane, benzene, toluene, xylene, chloroform, dichloromethane, 1,2-dichloroethane, 1,2-dichloropropane, 1,1,2-trichloroethane, 1,1,1-trichloroethane, trichloroethylene, tetrachloroethane, tetrahydrofuran, dioxolan, dioxane, methanol, ethanol, butanol, isopropanol, ethyl acetate, butyl acetate, dimethylsulfoxide and methyl cellosolve can be cited as the solvent or dispersion medium to be used for forming the intermediate layer, charge generation layer and charge transfer layer. A solvent gentle for the global environment such as tetrahydrofuran and methyl ethyl ketone is preferably used. These solvents may be used solely or in a state of mixture of two or more kinds of them.

As the coating method for preparing the organic photoreceptor of the invention, a coating method such as a slide hopper coating, a dipping coating and a spray coating are applicable.

Among the above coating liquid supplying type coating apparatus, the coating method using a slide hopper type coating apparatus is most suitable for coating a dispersion using the above low-boiling solvent. When a coating is carried out on a cylindrical photoreceptor, the coating by the use of a circular slide hopper coating apparatus described in JP-A 58-189061 is preferable.

It is preferable that an antioxidant is contained in the surface layer of the photoreceptor relating to the invention. The surface layer tends to be oxidized by reactive gas such as NOₓ and ozone generated on the occasion of charging of the photoreceptor so as to cause blurring of image, but the blurring of image can be prevented by the coexistence of the antioxidant. The antioxidant is typically a substance capable of preventing or inhibiting the affect of oxygen in the presence of light, heat or electric discharge to an auto-oxidizable material existing in the photoreceptor or on the surface thereof.

In the image forming method of the invention, a polymerized toner is preferably used in the developer to be used in the developing means. A suitable electrophotographic image having further high sharpness can be obtained by the use of the polymerized toner having uniform shape and particle size distribution in combination with the organic photoreceptor of the invention.

The electrostatic latent image formed on the photoreceptor of the invention is made visible into a form of toner image by the development. As the toner relating to the invention is preferably a polymerized toner produced by a polymerization method from the viewpoint of that the stable size distribution can be obtained though the toner to be used for the development may be a crushed toner or polymerized toner.

The polymerized toner is a toner in which formation of the binder resin and making the shape of the toner are performed by polymerization of a raw material monomer and a chemical treatment after the polymerization according necessity. In concrete, the toner is formed by a polymerization reaction such as suspension polymerization and emulsion polymerization and a process for fusing the particles carried out after the polymerization according to necessity.

The volume average particle diameter, namely the 50% volume particle diameter (Dv50 is preferably from 2 to 9 μm, and more preferably from 3 to 7 μm. The resolution can be raised by making the volume average particle into such the range. Moreover, the amount of the fine toner particle can be reduced in the above particle size range so that the reproducibility of dot image is improved for long duration and an image with high sharpness and stability can be obtained.

The toner relating to the invention may be used as a one-component developer or in a state of two-component developer.

When the toner is used as the one-component developer, the toner can be used for both of a non-magnetic one-component developer and a magnetic one-component developer containing magnetic particles of approximately from 0.1 to 0.5 μm in the toner particle.

The toner may be used for two-component developer by mixing with a carrier. In such the case, known material such as iron, ferrite, magnetite, and an alloy of such the metal and aluminum or lead can be used as the magnetic carrier particle. Ferrite is particularly preferred. The volume average diameter of the magnetic particles is preferably from 15 to 100 μm and more preferably from 25 to 80 μm.

The volume average diameter of the magnetic carrier particles can be measured by typically a laser diffraction particle size distribution measuring apparatus having a wet type dispersing means HELOS manufactured by Sympatec GmbH.

A carrier composed of magnetic particle coated with resin and a resin dispersion type carrier composed of magnetic particle dispersed in resin is preferable. For example, an olefin type resin, styrene type resin, a styrene-acryl type resin, a silicone type resin, an ester type resin or a fluorine-containing polymer are used as the coating resin though the resin is not specifically limited. Resins such as a styrene-acryl type resin, a polyester resin, a fluororesin and a phenyl resin are usable.

The image forming apparatus using the organic photoreceptor of the invention is described below.

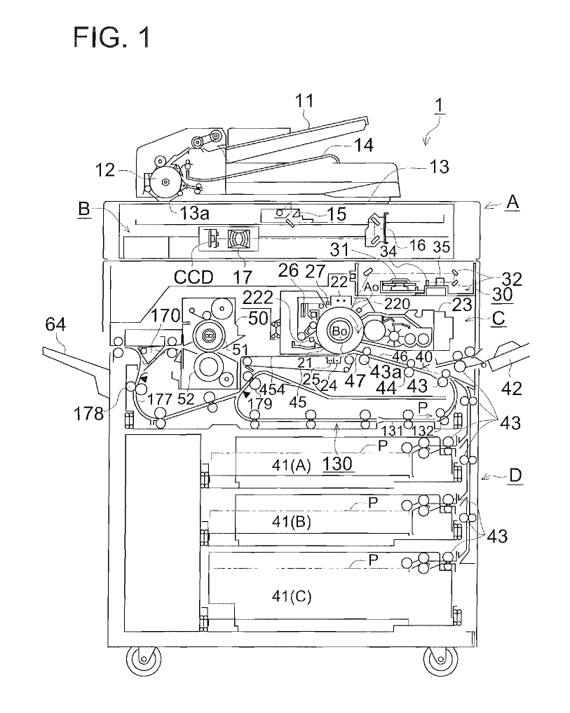

The image forming apparatus 1 shown in FIG. 1 is an image forming apparatus by a digital system, which is constituted by an image reading portion A, an image processing portion B, an image forming portion C and an image transfer paper conveying portion D as a recording paper conveying means.

An automatic original conveying means for automatically conveying the original image is provided on the image reading portion A and the original placed on an original placing stand 11 is conveyed one by one by an original conveying roller 12 and the image is read at the reading position 13a. The original is discharged after image reading onto an original discharging tray 14 by an original conveying roller 12.

On the other hand, when the original is placed on a platen glass 13, the image of the original is read by reading action at a rate of v by a first mirror unit 15 composed of a lighting lamp and a first mirror and motion of a second mirror unit 16 composed of a second mirror and a third mirror arranged in V-shaped position at a rate of v/2 in the same direction.

The read image is focused through a projection lens 17 on the light receiving face of an image taking element CCD as a line sensor. The line-shaped optical image focused on the image taking element CCD is successively converted by photoelectric conversion into electric signals (luminance signals) and subjected to A/D conversion. And then the signals are subjected to density conversion and filtering treatment and memorized at once in a memory element.

In the image forming portion C, a drum-shaped photoreceptor 21 as an image carrier, and a charging means (charging process) 22 for charging the photoreceptor 21, a potential detecting means 220 for detecting the surface potential of the photoreceptor after charging, a developing means (developing process) 23, a transfer conveying belt 45 as a transferring means (transfer process), a cleaning device (cleaning process) 26 for the photoreceptor 21 and PCL (pre-charging lamp) 27 as a photo charge removing means each arranged around the photoreceptor in the acting order, are installed in a state of an image forming unit. A reflective density detecting means 222 for measuring a patch image developed on the photoreceptor 21 is provided on the down stream side of the developing means 23. The organic photoreceptor of the invention is used as the photoreceptor 21 which is driven for rounding in the clockwise direction in the drawing.

The rotating photoreceptor 21 is uniformly charged by the charging means 22 and then image wise exposed to light according to the image signals readout from the memory of the image processing portion B by the exposing optical system as the imagewise exposing means (imagewise exposing process) 30. In the exposing optical system of the imagewise exposing means 30 as a writing means, the light beam from a laser diode, not displayed in the drawing, as the light source is conducted through a rotating polygon mirror 31, a fθ lens 34, a cylindrical lens 35 and reflected by a reflecting mirror 32 for main-scanning. The imagewise exposure is given to the photoreceptor at the position Ao and an electrostatic latent image is formed by the rotation (subsidiary-scanning direction) of the photoreceptor 21. In an example of embodiment of the invention, the exposure is given at the letter portion for forming the latent image.

In the image forming apparatus of the invention, a semiconductor laser or a light emission diode each emitting light having wavelength of from 350 to 500 nm is used as the light source for imagewise exposing. A high resolution electrophotographic image of from 600 dpi (dpi: dot number per inch, 2.54 cm) to 2,500 dpi can be obtained by using such the light source and narrowing the exposing dot diameter for writing in the primary-scanning direction of writing to 10 to 50 μm for carrying out digital exposure on the organic photoreceptor.

The above dot diameter is the length of the exposing light beam of the region in which the intensity of light is not less than 1/e² of the peak intensity along the main-scanning direction (Ld: the length measured at the position where the length is the maximum).

A canning system using the semiconductor laser and a solid state scanner of LED are usable for emitting the light beam, and Gauss distribution and Lorenz distribution are applicable as the light intensity distribution, and the exposing dot diameter is defined by the region of not less than 1/e² of the peak intensity in any cased.

The electrostatic latent image formed on the photoreceptor 21 is reversely developed by the developing means 23; thus a visible toner image is formed on the surface of the photoreceptor 21.

In the image recording paper conveying portion B, paper supplying units 41(A), 41(B) and 41(C) as image recording paper storing means in each of which image recording paper sheets P different in the size are stacked are provided under the image forming unit, and a hand paper supplying unit 42 for supplying paper by hand is provided on one side of the paper. The image recording paper P selected from the above paper storing means is conveyed through a conveying rout 40 and temporarily stopped by a paper supplying resist roller 44 for correcting the lean and position of the paper. And then the image recording paper is guided by the conveying rout 40, a roller before image transfer 43a, a paper supplying rout 46 and a guiding plate 25 to a transferring position Bo. At the position Bo, the toner image on the photoreceptor 21 is transferred onto the image recording paper P by a transferring electrode 24 and a separation electrode 25 while the paper is conveyed by a transfer conveying belt 454 of a transfer conveying device 45. The image recording paper P is separated from the surface of photoreceptor 21 and conveyed to a fixing means 50 by the conveyer belt 45.

The fixing means 50 has a fixing roller 51 and a pressing roller 52 and the toner is fixed by pressing and heating on the occasion of passing the image recording paper P between the fixing roller 51 and the pressing roller 52. The image recording paper P is output onto a paper receiving tray 64 after fixation of the toner image.

The situation of image formation on one side of the image recording paper is described in the above. In the case of both face copying, a paper exhausting changing member 170 is turned and a recording paper guide 177 is opened so that the recording paper is conveyed in the direction shown by the arrow of broken line.

The recording paper P is conveyed in the lower direction by a conveying device 178 and switch-backed by a recording paper reversing member 179 so that the tail end of the paper becomes to the forefront of the paper and conveyed into a paper supplying unit for both face copying 130.

The recording paper is conveyed in the supplying direction along the conveying guide 131 in the paper supplying for both side copying 130, and supplied into the conveying rout 40 by a paper supplying roller 132.

The image recording paper P is conveyed to the photoreceptor 21 and a toner image is transferred onto the back side of the paper P and the paper P is output onto the paper output tray 64 after fixation of the toner image by the fixing means 50.

In the image forming apparatus of the invention, the constituting members such as the photoreceptor, developing device and cleaning device may be unified into a processing cartridge which is installed in the apparatus so as to be freely installed and released. At least one of the charging device, imagewise exposing device, developing device, transferring or separation device and cleaning device may be held together with the photoreceptor to form the processing cartridge which may be installed in the main body of the apparatus by a guiding means such as a rail so that the unit can be freely installed and released.

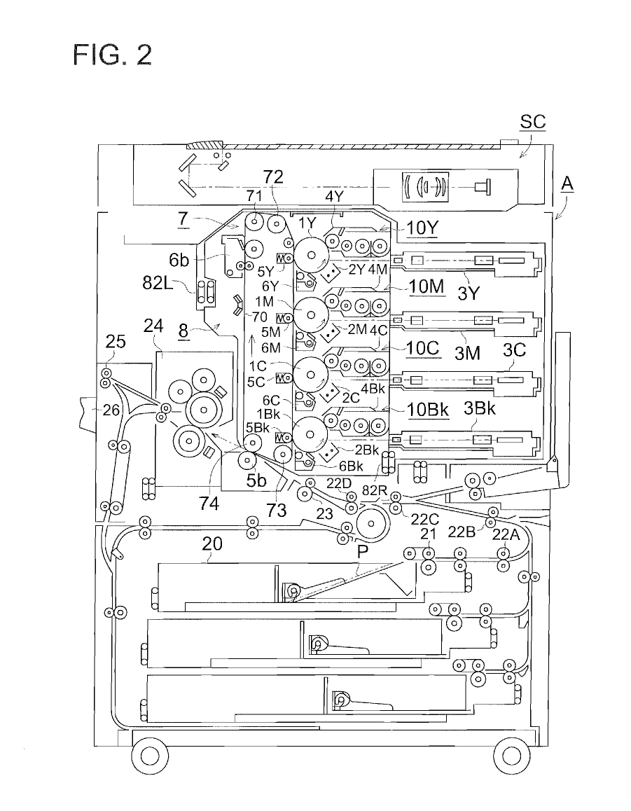

FIG. 2 displays a cross section of a color image forming apparatus showing an embodiment of the invention.

The color image forming apparatus is one called as a tandem type color image forming apparatus, which comprises four image forming units 10Y, 10M, 10C and 10Bk, an endless belt-shaped intermediate transferring member unit 7, paper supplying means 21 and a fixing means 24. Upper portion of the main body A of the image forming apparatus, an original image reading apparatus SC is provided.

The image forming unit 10Y for forming a yellow image has a drum-shaped photoreceptor 1Y as a primary image carrier, and a charging means (charging process) 2Y, an exposing means (exposing process) 3Y, a developing means (developing process) 4Y, a primary transfer roller 5Y as a primary transfer means (primary transfer process) and a cleaning means 6Y each arranged around the photoreceptor 1Y. The image forming unit 10M for forming a magenta image has a drum-shaped photoreceptor 1M as a primary image carrier, a charging means 2M, an exposing means 3M, a developing means 4M, a primary transfer roller 5M as a primary transfer means and a cleaning means 6M. The image forming unit 10C for forming a cyan image has a drum-shaped photoreceptor 1C as a primary image carrier, a charging means 2C, an exposing means 3C, a developing means 4C, a primary transfer roller 5C as a primary transfer means and a cleaning means 6C. The image forming unit 10Bk for forming a black image has a drum-shaped photoreceptor 1Bk as a primary image carrier, a charging means 2Bk, an exposing means 3Bk, a developing means 4Bk, a primary transfer roller 5Bk as a primary transfer means and a cleaning means 6Bk.

The above four image forming units 10Y, 10M, 10C and 10Bk are each constituted by the drum-shaped photoreceptors 1Y, 1M, 1C and 1Bk each placed at the central portion of the init, the charging means 2Y, 2M, 2C and 2Bk, the imagewise exposing means 3Y, 3M, 3C and 3Bk, the developing means 4Y, 4M, 4C and 4Bk, the cleaning means 6Y, 6M, 6C and 6Bk, respectively.

The image forming units 10Y, 10M, 10C and 10Bk are the same with each other except that the colors of the toner are different. Therefore, the unit is described in detail about the image forming unit 10Y for example.

In the image forming unit 10Y, the charging means 2Y, hereinafter also referred to as charging means 2Y or charging device 2Y, the exposing means 3Y, the developing means 4Y, and the cleaning means 6Y, hereinafter also referred to as cleaning means 6Y or cleaning blade 6Y, are arranged around the photoreceptor 1Y, and a yellow toner image is formed on the photoreceptor drum 1Y. In this embodiment, the photoreceptor 1Y, charging means 2Y, developing means 4Y and cleaning means 6Y are provided to form a unified state.

The charging means 2Y is a means for uniformly charging the photoreceptor drum 1Y and a corona discharging type charging device 2Y is used in this embodiment.

The imagewise exposing means 3Y is a means for forming an electrostatic image corresponding to a yellow image by giving light exposure to the uniformly charged photoreceptor drum 1Y according to yellow image signals. As the exposing means, one constituted by LED elements arranged in an ally shape and an image focusing element (commercial name: Selfoc lens) or a laser optical system is used.

In the image forming apparatus of the invention, the constituting members such as the photoreceptor, developing device and cleaning device may be unified into a processing cartridge which is installed in the apparatus so as to be freely installed and released. At least one of the charging device, imagewise exposing device, developing device, transferring or separation device and cleaning device may be held together with the photoreceptor to form a processing cartridge (image forming unit) which may be installed to the main body of the apparatus by a guiding means such as a rail so that the unit can be freely installed and released.

The endless belt-shaped intermediate transfer unit 7 is rounded on plural rollers, which has an endless belt-shaped intermediate transferring member 70 as an endless belt-shaped semi-electroconductive secondary image carrier.

Color images formed by the image forming units 10Y, 10M, 10C and 10Bk are each successively transferred onto the roundly moving endless belt-shaped intermediate transferring member 70 by the primary transferring rollers 5Y, 5M, 5C and 5Bk to form a synthesized color image. The recording material (support for carrying the fixed final image such as common paper and transparent sheet) as the recording material P stored in the paper supplying cassette 20 is supplied by a paper supplying means 21 and conveyed to a secondary transfer roller 5b as the secondary transfer roller through plural intermediate rollers 22A, 22B, 22C, 22D and a resist roller 23, and the color image is secondarily transferred at once on the recording material P. The recording material P on which the color image is transferred is fixed by a fixing means 24 and held conveyed by a paper outputting roller 25 so as to be placed on an output paper tray 26. The transferring supports for supporting the toner image formed on the photoreceptor such as the intermediate transfer member and the recording material are generally referred to as a transferring medium.

On the other hand, the endless belt-shaped intermediate transferring member 70 releases the recording material P by curvature after transferring the color image to the recording material P by the secondary transfer roller 5b as the secondary transfer means and then the remaining toner is removed by a cleaning means 6b.

The primary transfer roller 5Bk is constantly contacted to the photoreceptor 1Bk during the image forming processing. The other primary transfer rollers 5Y, 5M and 5C are each contacted to the corresponding photoreceptor 1Y, 1M and 1C, respectively, only when the color image is formed.

The secondary transfer roller 5b is contacted to the endless belt-shaped, intermediate transferring member 70 only when the secondary transfer is performed by passing the recording material P at this position.

The case 8 is installed through supporting rails 82L and 82R so that the case can be pulled out from the main body A.

The case 8 contains the image forming units 10Y, 10M, 10C and 10Bk, and the endless-shaped intermediate transfer member unit 7.

The image forming units 10Y, 10M, 10C and 10Bk are lined in the vertical direction. The endless belt-shaped intermediate transfer unit 7 is arranged on the left side of the photoreceptors 1Y, 1M, 1C and 1Bk. The endless belt-shaped intermediate transfer unit 7 is constituted by the endless belt-shaped intermediate transfer member 70 rotatable around rollers 71, 72, 73 and 74, primary transfer roller 5Y, 5M, 5C and 5Bk and the cleaning means 6b.

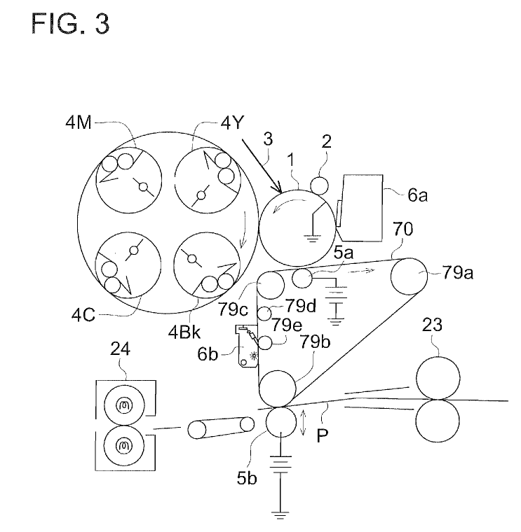

FIG. 3 is a cross section of a color image forming apparatus using the organic photoreceptor of the invention, which is a copying machine or a laser beam printer having a charging means, an exposing means, plural developing means, a transfer means, a cleaning means and an intermediate transfer member each provided around the organic photoreceptor. An elastic material having medium resistivity is used for the belt-shaped intermediate transfer member 70.

1 is a rotatable drum-shaped photoreceptor which is repeatedly usable as an image forming member and driven for rotating in anticlockwise direction as shown by the arrow at a designated circumference speed.

In the course of the rotation, the photoreceptor 1 is uniformly charged at a designated polarity and potential by the charging means 2 (charging process) r and receives image wise exposure by main-scanning by a laser beam correspondingly modulated by time serial electric digital signals of the image information from the imagewise exposing means 3 (imagewise exposure process), which is not displayed in the drawing to form an electrostatic latent image corresponding to the objective color component image (color information) of yellow (Y).

After that, the electrostatic latent image is developed by a yellow toner by a yellow color developing device 4 (development process) as the developing means of yellow (Y) to form a yellow toner image as a first color. The action of second to fourth developing means 4M, 4C and 4Bk (a magenta color developing device, cyan color developing device and black color developing device) are turned off and do not affect to the photoreceptor at this time so that the yellow toner image of the first color is not influenced by the second to fourth developing devices.

The intermediate transfer member 70 is put up by rollers 79a, 79b, 79c, 79d and 79e and driven and rotated in the clockwise direction at the same circumference speed as that of the photoreceptor 1.

The first color of yellow toner image formed and carried on the photoreceptor 1 is successively transferred onto the outer surface of the intermediate transfer member 70 (primary transfer) in the course of passing the nipping portion of the photoreceptor 1 and the intermediate transfer member 70 by a electric field applied to the intermediate transfer member 70 from a primary transfer roller 5b.

The surface of the photoreceptor 1 is cleared by a cleaning device 6a after completion of the transfer of the first color of yellow toner image corresponding to the intermediate transfer member 70.

Thereafter, the magenta toner image as the second color, the cyan toner image as the third color and the black toner image as the fourth color are successively superposed on the intermediate transfer member 70 in the same manner to form a color toner image.

A secondary transfer roller 5b is separably provided under side of the intermediate transfer member 70 facing in parallel with a facing secondary transfer roller 79b.

The primary transfer bias for successively transferring the first to fourth color toner images as superposed from the photoreceptors 1Y, 1M, 1C and 1K to the intermediate transfer member 70 has reverse polarity to that of the toner and applied by a bias power source. The applying voltage of that is within the range of from +100 V to +2 kV for example.

The secondary transfer roller 5b and the intermediate transfer member cleaning means 6b can be released from the intermediate transfer member 70 in the primary transfer process of toner images of the first to third color on the photoreceptors 1Y, 1M and 1C to the intermediate transfer member 70.

The transfer of the color toner image transferred and superposed on the belt-shaped intermediate transfer member 70 to the recording material P as the secondary image carrier is carried out by that the secondary transfer roller 5b is contacted to the belt of the intermediate transfer member 70 and the recording material P is supplied at the designated timing to the nipping portion formed by contacting the intermediate transfer member 70 and the secondary transfer roller 5b through a pair of paper supplying resist rollers 23 and a recording paper guide. Secondary transfer bias is applied to the secondary transfer roller 5b from a bias power source. The superposed color toner image is transferred (secondary transfer) onto the recording material P as the secondary image carrier from the intermediate transfer member 70 by the secondary transfer bias. The recording material P on which the toner image is transferred is introduced into the fixing means 24 and thermally fixed.

The image forming apparatus is generally suitable for electrophotographic apparatuses such as electrophotographic copiers, laser printers, LED printers and liquid crystal shutter type printers. Furthermore, the image forming apparatus can be widely applied for apparatuses utilizing electrophotography such as displaying, recording, light printing, plate making and facsimile apparatuses.

### EXAMPLES

The invention is described in detail referring examples. In the followings, “part” means “parts by weight”.

### Example 1

### Preparation of Photoreceptor 1

Photoreceptor 1 was prepared as follows.

An electroconductive substrate was prepared by finishing the surface of a cylindrical aluminum substrate so that the ten point-surface roughness Rz was made to 1.5 μm.

Intermediate Layer 1

The following intermediate layer coating liquid was coated on the above electroconductive substrate by a dipping coating method and dried at 120° C. for 30 minutes to form an intermediate layer 1 having a dry thickness of 1.0 μm.

The following dispersion for intermediate layer was diluted by two times by the same solvent and filtered by RIGIMESH filter (nominal filtering accuracy: 5 μm, pressure: 50 kPa) manufactured by Nihon Pall Ltd., after standing for one night to prepare an intermediate layer coating liquid.

### (Preparation of Intermediate Layer Coating Liquid)

| | |
|---|---|
| Binder resin: Exemplified Polyamide N-1 | 1 part |
| | (1.00 parts by volume) |
| N-type semiconductor particle: Rutile type | 3.5 parts |
| titanium oxide A1 (primary particle diam- | (1.0 part by volume) |
| eter 35 nm, surface treated by 5% by weight | |
| of titanium oxide of copolymer of methyl- | |
| hydrogen siloxane and dimethylsiloxane in a | |
| mole ratio of 1:1) | |
| Mixture of ethanol, n-propyl alcohol and | 10 parts |
| THF in weight ratio of 45/20/30 | |

The above composition was mixed and dispersed by butch system for 10 hours by a sand mill dispersing machine to prepare the dispersion for intermediate layer.

(Charge Generation Layer CGL)

| | |
|---|---|
| Charge generation material (CGM): Multi-step sublimation | 24 parts |
| purified CGM-13 having a mass reduction ratio of 0.4% | |
| Poly(vinyl butyral) resin, S-Lec BL-1 (manufactured by | 12 parts |
| Sekisui Chemical Co., Ltd.) | |
| Mixture of 2-butanone and cyclohexanone in a volume | 300 parts |
| ratio of 4:1 | |

The above composition was mixed and dispersed by a sand mill employing glass beads (Highbea D24, manufactured by Ohara Inc.), with disk rotation rate at 500 rpm for 5 hours maintaining the dispersion liquid temperature at 25±5° C. to prepare a charge generation layer coating liquid. Average particle diameter of the CGM was 0.07 mm, measured by a ctrifugal sedimentation method employing CAPA-700, manufactured by Horiba Ltd. The coating liquid was coated by the dipping coating method to form a charge generation layer having a dry thickness of 0.5 μm on the intermediate layer.

(Charged Transfer Layer)

| | | |
|---|---|---|
| Charge transfer material (CTM): The following CTM-1 | 225 | parts |
| Polycarbonate (Z300 manufactured by Mitsubishi Gas | 300 | parts |
| Chemical Co., Inc.) | | |
| Antioxidant (The following AO-1) | 6 | parts |
| A mixture of THF and toluene (3:1 in volume) | 2,000 | parts |
| Silicone oil (KF-54 manufactured by Shin-Etsu Chemical | 1 | part |
| Co., Ltd.) | | |

The above composition was mixed and dissolved to prepare a charge transfer layer coating liquid. The coating liquid was coated on the charge generation layer by the dipping coating method and dried at 110° C. for 70 minutes to form charge transfer layer 1 having a dry thickness of 20.0 μm. Thus Photoreceptor 1 was prepared

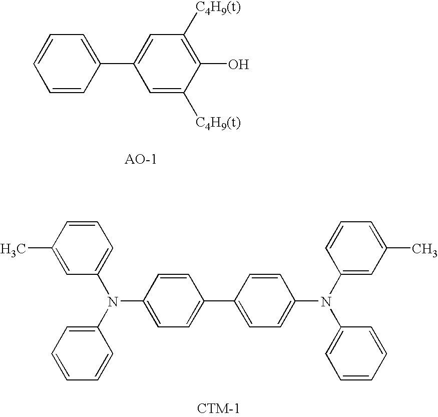

### Preparation of Photoreceptors 2 to 5

Photoreceptors 2 to 5 were prepared in the same manner as in Photoreceptor 1 except that the CGM-13 was replaced by each of the CGMs listed in Table 1 which are each subjected to the multi-step sublimation purification (Purification Example 1) the same as that in CGM-13 used in Photoreceptor 1.

### Preparation of Photoreceptor 6

Photoreceptor 6 was prepared in the same manner as in Photoreceptor 1 except that the CGM-13 was replaced by CGM purified only by the first step of Purification Example 1.

### Preparation of Photoreceptor 7

Photoreceptor 7 was prepared in the same manner as in Photoreceptor 1 except that CGM-13 synthesized by Synthesis Example 1 is used without any purification.

### Preparation of Photoreceptor 8

Photoreceptor 8 was prepared in the same manner as in Photoreceptor 1 except that CGM was replaced by a pyranthrone compound CGM-31 of the following formula, commercially available in the name of Pallogen Red L 3530, by BASF.

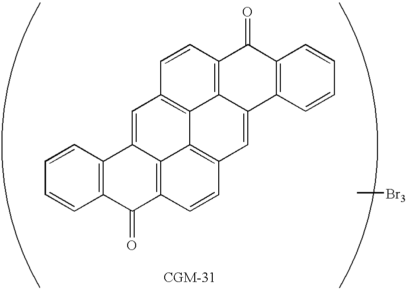

<<Evaluation>>

The photoreceptors prepared as the above were evaluated as follows by using an electrostatic copy paper testing apparatus EPA-8100 manufactured by Kawaguchi Electric Works Co., Ltd.

(Sensitivity)

The photoreceptor was charged by a corona charging device so that the surface potential of the photoreceptor is made to −700 V and then exposed to monochromatic light of 400 nm, and the light amount necessary for attenuating the surface potential by −350 V was measured for determining the sensitivity (E_{1/2}).

The sensitivities at 450 nm and 500 nm were also measured.

(Repeating Usability)

The initial dark portion potential (Vd) and the initial light portion potential (V1) were each set at −700 V and −200 V, respectively, and the charging and exposing were repeated for 3,000 times using monochromatic light of 450 nm, and the variations in the Vd and V1 (ΔVd and ΔV1) were measured.

The results of the above evaluation are listed in Table 1.

In the table, “+” represents lowering in the potential and “−” represents rising in the potential.

(Image Evaluation)

A digital copying machine Sitios 7085, manufactured by Konica Minolta Business Technologies Inc., was used for the evaluation, which is modified so that a semiconductor laser emitting light of 450 nm was used as the light source and the exposure of 1,200 dpi was carried out by using a light beam of 30 μm and the processing speed was changed to 500 mm/sec. Photoreceptors 1 to 9 were respectively installed into the testing machine and evaluated. The evaluation items and the evaluation norms are shown below.

Evaluation of One-Dot Line

A one-dot line and a black solid image were formed on an A4 size white paper sheet and evaluated according to the following norms.

A: The one-dot line was reproduced in continuous and the density of the black solid image was 1.2 or more. (Good)

B: The one-dot line was reproduced in continuous but the density of the black solid image was less than 1.2 and not less than 1.0. (No problem was caused in practical use.)

C: The one-dot line was brokenly reproduced or the density of the black solid image was less than 1.0 even when the one-dot line was reproduced in continuous. (A problem was caused in practical use.)

Evaluation of Two-Dot Line

A two-dot line was formed on a black solid background was formed and evaluated according to the following norms.

A: The two-dot line was reproduced in continuous and the density of the black solid image was 1.2 or more. (Good)

B: The two-dot line was reproduced in continuous but the density of the black solid image was less than 1.2 and not less than 1.0. (No problem was caused in practical use.)

C: The two-dot line was brokenly reproduced or the density of the black solid image was less than 1.0 even when the one-dot line was reproduced in continuous. (A problem was caused in practical use.)

The above image density was measured by Macbeth RD-918, manufactured by Macbeth and represented by the relative reflective density when the reflective density of the paper was set at zero. The results are shown in Table 1.

TABLE 1

| | | | | | | | | | image evaluation | |
|---|---|---|---|---|---|---|---|---|---|---|
| | Charge generation | | | Sensitivity E_{1/2} | | | | | Repro- | Repro- |
| Photo- | material | Purification treatment | Mass | (μJ/cm²) | | | Repeating | | ducibility | ducibility |
| receptor | Exemplified | of charge generation | reduction | 400 | 450 | 500 | usability (V) | | of one-dot | of two-dot |
| No. | compound No. | material | ratio (%) | nm | nm | nm | ΔVd | ΔVl | line | line |
| 1 | CGM-13 | *1 | 0.40 | 0.26 | 0.22 | 0.13 | −10 | +10 | A | A |
| 2 | CGM-2 | *1 | 1.80 | 0.35 | 0.30 | 0.24 | −20 | +20 | B | B |
| 3 | CGM-10 | *1 | 0.19 | 0.29 | 0.25 | 0.18 | −10 | +10 | A | A |
| 4 | CGM-16 | *1 | 0.35 | 0.30 | 0.27 | 0.24 | −10 | +10 | A | A |
| 5 | CGM-24 | *1 | 1.20 | 0.27 | 0.24 | 0.20 | −20 | +15 | B | A |
| 6 | CGM-13 | One-step sublimation | 2.07 | 0.95 | 0.87 | 0.57 | −50 | +40 | C | C |
| | | purification | | | | | | | | |
| 7 | CGM-13 | None | 4.45 | 1.38 | 1.26 | 1.11 | −80 | +110 | C | C |
| 8 | CGM-31 | None | 3.14 | 1.33 | 1.17 | 1.09 | −75 | +95 | C | C |

*1: Multi-step sublimation purification (The condition the same as that in Purification Example 1

As is cleared in Table 1, the organic photoreceptors 1 to 5 using the charge generation material of the pyranthrone type compound represented by Formula 1 purified by the multi-step sublimation display superior sensitivity and repeating usability to light of 400 to 500 nm such as short wavelength laser light and the reproducibility is also superior in the reproducibility of one-dot line and two-dot line by short wavelength laser light of 450 nm. On the other hand, comparative Photoreceptor 6 using the one-step sublimation purified charge generation material and Photoreceptor 7 using the charge generation material without purification as well as the pyranthrone compound on the market without purifying process were relatively inferior to Photoreceptors 1 to 5 of the invention in the sensitivity and the repeating usability and degradation in the reproducibility of the one-dot line in the image evaluation is large.

### Preparation of Photoreceptors 11 to 16

Photoreceptors 11 to 16 were prepared in the same manner as in Photoreceptor 1 except that the CGM purified by the multi-step sublimation purification was replaced by ones purified by the separation sublimation purification as shown in Table 2. In Photoreceptor 12, the purification condition of the CGM-13 was changed so as to change the length of the temperature gradient to 0.5 m from 1.0 m; the range of the temperature of from about 480° C. to about 20° C. was not changed.

<<Evaluation 2>>

Photoreceptors 11 to 16 were evaluated in the same manner as in Photoreceptor 1. The results are listed in Table 2.

TABLE 2

| | Charge generation | | | Sensitivity E_{1/2} | | | | | | |
|---|---|---|---|---|---|---|---|---|---|---|
| Photo- | material | Purification treatment | Mass | (μJ/cm²) | | | Repeating | | | |
| receptor | Exemplified | of charge generation | reduction | 400 | 450 | 500 | usability (V) | | image evaluation | |
| No. | compound No. | material | ratio (%) | nm | nm | nm | ΔVd | ΔVl | *1 | *2 |
| 11 | CGM-13 | Separation sublimation | 0.24 | 0.22 | 0.20 | 0.13 | −10 | +5 | A | A |
| | | purification (The same | | | | | | | | |
| | | condition as that in | | | | | | | | |
| | | Purification Example 2) | | | | | | | | |
| 12 | CGM-13 | *3 | 0.35 | 0.26 | 0.23 | 0.20 | −10 | +10 | A | A |
| 13 | CGM-2 | Separation sublimation | 0.75 | 0.35 | 0.31 | 0.23 | −10 | +10 | A | A |
| | | purification (The same | | | | | | | | |
| | | condition as that in | | | | | | | | |
| | | Purification Example 2) | | | | | | | | |
| 14 | CGM-10 | Separation sublimation | 0.25 | 0.27 | 0.24 | 0.20 | −10 | +10 | A | A |
| | | purification (The same | | | | | | | | |
| | | condition as that in | | | | | | | | |
| | | Purification Example 2) | | | | | | | | |
| 15 | CGM-16 | Separation sublimation | 0.37 | 0.31 | 0.26 | 0.22 | −10 | +10 | A | A |
| | | purification (The same | | | | | | | | |
| | | condition as that in | | | | | | | | |
| | | Purification Example 2) | | | | | | | | |
| 16 | CGM-24 | Separation sublimation | 1.15 | 0.28 | 0.25 | 0.21 | −15 | +15 | B | A |
| | | purification (The same | | | | | | | | |
| | | condition as that in | | | | | | | | |
| | | Purification Example 2) | | | | | | | | |

*1: Reproducibility of one-dot line
*2: Reproducibility of two-dot line
*3: Separation sublimation purification (The same condition as that in Purification Example 2 except that the length of temperature gradient was changed to 0.5 m.)

As is cleared in Table 2, the organic photoreceptors 11 to 16 using the charge generation material of the pyranthrone type compound represented by Formula 1 purified by the separation sublimation display superior sensitivity and repeating usability to light of 400 to 500 nm such as short wavelength laser light and the reproducibility is also superior in the reproducibility of one-dot line and two-dot line by short wavelength laser light of 450 nm.

### Preparation of Photoreceptors 21 to 27

Photoreceptors 21 to 27 were prepared in the same manner as in Photoreceptor 1 except that the multi-step sublimation purification treatment of CGM-13 in Photoreceptor 1 was changed to the heat treatment in the high-boiling solvent, the solvent and the heating condition are shown in Table 3, and the exemplified compounds having the mass reduction ratio described in Table 3 were used.

### Preparation of Photoreceptor 28

Photoreceptor 28 was prepared in the same manner as in Photoreceptor 1 except that the multi-step sublimation of CGM-13 in Photoreceptor 1 was changed to the heat treatment in toluene solvent.

<<Evaluation 3>>

Photoreceptors 21 to 28 were evaluated in the same manner as for Photoreceptor 1 in Evaluation 1. The Results rare listed in Table 3.

TABLE 3

| | | Purification treatment | | | | | | | | |
|---|---|---|---|---|---|---|---|---|---|---|
| | Charge generation | of charge generation | | Sensitivity E_{1/2} | | | | | | |
| Photo- | material | material | Mass | (μJ/cm²) | | | Repeating | | | |
| receptor | Exemplified | (Kind of solvent and | reduction | 400 | 450 | 500 | usability (V) | | image evaluation | |
| No. | compound No. | treatment temperature) | ratio (%) | nm | nm | nm | ΔVd | ΔVl | *1 | *2 |
| 21 | CGM-13 | Heat treatment in high- | 0.68 | 0.28 | 0.24 | 0.21 | −10 | +10 | A | A |
| | | boiling solvent | | | | | | | | |
| | | (Nitrobenzene; 200° C.) | | | | | | | | |
| 22 | CGM-13 | Heat treatment in high- | 0.65 | 0.31 | 0.28 | 0.23 | −10 | +10 | A | A |
| | | boiling solvent (o- | | | | | | | | |
| | | dichlorobenzene; 170° C.) | | | | | | | | |
| 23 | CGM-13 | Heat treatment in high- | 0.51 | 0.26 | 0.23 | 0.18 | −10 | +5 | A | A |
| | | boiling solvent | | | | | | | | |
| | | (Quinoline; 200° C.) | | | | | | | | |
| 24 | CGM-2 | Heat treatment in high- | 1.30 | 0.36 | 0.31 | 0.26 | −20 | +15 | B | B |
| | | boiling solvent | | | | | | | | |
| | | (Nitrobenzene; 200° C.) | | | | | | | | |
| 25 | CGM-10 | Heat treatment in high- | 0.89 | 0.28 | 0.25 | 0.22 | −10 | +10 | A | A |
| | | boiling solvent | | | | | | | | |
| | | (Nitrobenzene; 200° C.) | | | | | | | | |
| 26 | CGM-16 | Heat treatment in high- | 0.77 | 0.30 | 0.27 | 0.23 | −10 | +10 | A | A |
| | | boiling solvent | | | | | | | | |
| | | (Nitrobenzene; 200° C.) | | | | | | | | |
| 27 | CGM-24 | Heat treatment in high- | 1.10 | 0.33 | 0.30 | 0.27 | −20 | +10 | B | A |
| | | boiling solvent | | | | | | | | |
| | | (Nitrobenzene; 200° C.) | | | | | | | | |
| 28 | CGM-13 | Heat treatment in high- | 2.85 | 0.76 | 0.72 | 0.70 | −60 | +50 | C | C |
| | | boiling solvent | | | | | | | | |
| | | (Toluene; 110° C.) | | | | | | | | |

*1: Reproducibility of one-dot line
*2: Reproducibility of two-dot line

As is cleared in Table 3, the organic photoreceptors 21 to 27 using the charge generation material of the pyranthrone type compound represented by Formula 1 treated by heating in the high-boiling solvent display superior sensitivity and repeating usability to light of 400 to 500 nm such as short wavelength laser light and the reproducibility is also superior in that of one-dot line and two-dot line by short wavelength laser light of 450 nm. On the other hand, comparative Photoreceptor 28 using the charge generation material treated by heating in toluene was relatively inferior to Photoreceptors 21 to 27 of the invention in the sensitivity, the repeating usability and the in the reproducibility of the one-dot line and two-dot line.

### Preparation of Photoreceptors 31 to 35

Photoreceptors 31 to 35 were prepared in the same manner as in Photoreceptor 1 except that the multi-step sublimation purification of CGM-13 in Photoreceptor 1 was changed by the acid paste treatment described in Table 4, the purification condition of which was entirely the same as Purification Example 4 and the exemplified compounds having the mass reduction ratio described in Table 4 were used.

<<Evaluation 4>>

Photoreceptors 31 to 35 were evaluated in the same manner as for Photoreceptor 1. Results are listed in Table 4.

TABLE 4

| | Charge generation | Purification treatment | | Sensitivity E_{1/2} | | | | | | |
|---|---|---|---|---|---|---|---|---|---|---|
| Photo- | material | of charge generation | Mass | (μJ/cm²) | | | Repeating | | | |
| receptor | Exemplified | material | reduction | 400 | 450 | 500 | usability (V) | | image evaluation | |
| No. | compound No. | (Purification condition) | ratio (%) | nm | nm | nm | ΔVd | ΔVl | *1 | *2 |
| 31 | CGM-13 | Acid paste treatment | 0.76 | 0.28 | 0.24 | 0.21 | −10 | +10 | A | A |
| | | (Purification condition) | | | | | | | | |
| 32 | CGM-2 | Acid paste treatment | 0.88 | 0.38 | 0.34 | 0.30 | −10 | +10 | A | A |
| | | (Purification condition) | | | | | | | | |
| 33 | CGM-10 | Acid paste treatment | 0.78 | 0.29 | 0.26 | 0.23 | −10 | +15 | A | A |
| | | (Purification condition) | | | | | | | | |
| 34 | CGM-16 | Acid paste treatment | 1.02 | 0.30 | 0.28 | 0.24 | −20 | +15 | B | A |
| | | (Purification condition) | | | | | | | | |
| 35 | CGM-24 | Acid paste treatment | 0.95 | 0.29 | 0.24 | 0.22 | −10 | +10 | A | A |
| | | (Purification condition) | | | | | | | | |

*1: Reproducibility of one-dot line
*2: Reproducibility of two-dot line

As is cleared in Table 4, the organic photoreceptors 31 to 35 using the charge generation material of the pyranthrone type compound represented by Formula 1 treated by the acid paste treatment display superior sensitivity and repeating usability to light of 400 to 500 nm such as short wavelength laser light and the reproducibility is also superior in the reproducibility of one-dot line and two-dot line by short wavelength laser light of 450 nm.

1. An organic photoreceptor having a photosensitive layer provided on an electroconductive substrate wherein the photosensitive layer contains a pyranthrone compound represented by Formula 1 which has been subjected to purification processing so as to have a mass reduction ratio D_{400/450} between 400° C. and 450° C. of not more than 2.0% in a thermogravimetric analysis;
D_{400/450}={(G₄₀₀−G₄₅₀)/G_{i}}×100
wherein Gᵢ is a mass at 25° C., and G₄₀₀ and G₄₅₀ are each mass at 400° C. and 450° C. of the pyranthrone compound in the thermogravimetric analysis, respectively, 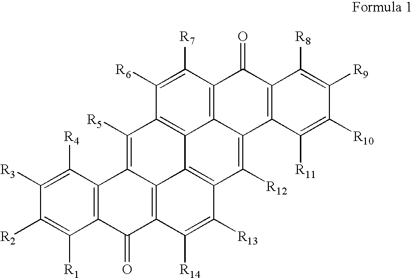
wherein R₁ through R₁₄ are each a hydrogen atom, an alkyl group, an alkoxy group, an aryl group, a halogen atom, a cyano group or a nitro group.

2. The organic photoreceptor of claim 1 wherein the purification process is multi-step sublimation purification.

3. The organic photoreceptor of claim 1 wherein the purification process is train sublimation purification.

4. The organic photoreceptor of claim 1 wherein the purification process is heat treatment in a high-boiling solvent.

5. The organic photoreceptor of claim 1 wherein the purification process is an acid paste treatment.

6. The organic photoreceptor of claim 1 wherein the mass reduction ratio D_{400/450} is not more than 1.8%.

7. The organic photoreceptor of claim 1 wherein the mass reduction ratio D_{400/450} is not more than 1.2%.

8. The organic photoreceptor of claim 1 wherein the mass reduction ratio D_{400/450} is not more than 1.0%.

9. The organic photoreceptor of claim 1 wherein R₁ through R₁₄ are each a hydrogen atom, an alkyl group, an alkoxy group, an aryl group or a halogen atom group.

10. An image forming method comprising the steps of
exposing for forming an electrostatic latent image on a photoreceptor by using a semiconductor laser or a light emission diode emitting light having a wavelength of from 350 to 500 nm as a writing light source, and
developing for developing the electrostatic latent image to form a toner image,
wherein the photoreceptor is the organic photoreceptor of claim 1.

11. A pyranthrone compound represented by Formula 1 which has been subjected to purification processing so as to have a mass reduction ratio D_{400/450} between 400° C. and 450° C. of not more than 2.0% in a thermogravimetric analysis;
D_{400/450}={(G₄₀₀−G₄₅₀)/Gᵢ}×100
wherein Gᵢ is a mass at 25° C. and G₄₀₀ and G₄₅₀ are each mass at 400° C. and 450° C. of the pyranthrone compound in the thermogravimetric analysis, respectively, 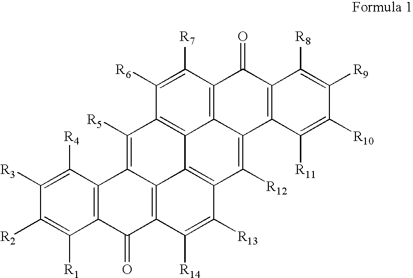
wherein R₁ and R₁₄ are each a hydrogen atom, an alkyl group, and alkoxy group, an aryl group, a halogen atom, a cyano group or a nitro group.
